(19) United States
(12) Patent Application Publication
(10) Pub. No.: US 2016/0101264 A1
(43) Pub. Date: Apr. 14, 2016

(54) CUTANEOUS CATHETER ANCHORING DEVICE AND METHOD OF STABILIZING A CATHETER SITE

(71) Applicant: Benjamin Bertram, Columbus, OH (US)

(72) Inventor: Benjamin Bertram, Columbus, OH (US)

(21) Appl. No.: 14/512,640

(22) Filed: Oct. 13, 2014

(51) Int. Cl. A61M 25/02

(52) U.S. Cl. CPC A61M 25/02; A61M 2025/0266

(57) ABSTRACT

A cutaneous catheter anchoring device and method to prevent occlusion of an indwelling peripheral intravenous catheter and to do so in a way that does not require immobilization of, or significant reduction in the range of motion of, the joint of the particular extremity. The device and method accomplish this by immobilizing multiple areas of the skin's surface relative to the indwelling catheter. The device is in close proximity to the indwelling catheter and maintains a smooth, rather than bent or creased, skin surface above the catheter thus preventing creasing or kinking of the catheter itself during times when the patient bends the extremity.

### CROSS-REFERENCE TO RELATED APPLICATIONS

Not applicable.

### STATEMENT REGARDING FEDERALLY SPONSORED RESEARCH OR DEVELOPMENT

Not applicable.

### TECHNICAL FIELD OF THE INVENTION

The present invention relates generally to anchoring devices for cutaneous catheters, particularly to devices for anchoring cutaneous intravenous catheters with a reduced degree of discomfort and loss of mobility in humans.

### BACKGROUND OF THE INVENTION

The securement of intravenous catheters in human medical patients represents a balancing of interests that have met with limited success to date. Optimally, an anchoring device will protect a catheter from dislodgment or disruption, but at the same time will have a minimal effect on patient mobility and comfort.

In the medical field today a common method of infusing fluids into the veins of a patient is through a peripheral intravenous catheter (or iv catheter) An iv catheter consists of a flexible plastic-like material and is basically in the form of a tube. It is inserted through the skin of a patient into a vein by way of a needle which is then retracted and discarded, leaving the tube-like catheter to indwell within the vein. The catheter terminates at the hub which is a more solid or inflexible structure. The hub is usually secured to the skin by a “stat-lock” which is an anchoring device which uses and adhesive base to and hub engaging means intended to prevent accidental catheter dislodgement. The stat-lock secured hub is then properly connected to iv tubing. The insertion point and a general surrounding area of skin is generally referred to as the “iv site.” The iv site includes the stat-lock and the engaged hub and is properly covered with and adhesive film dressing (also called window dressing, op-site, or tegaderm). This adhesive film dressing helps to maintain sterility of the site. The film's transparency allows visual inspection of the iv site by the nurse. The iv tubing eventually terminates into an elevated bag or bottle containing the therapeutic fluid prescribed for the patient. An iv pump is usually attached to the tubing to deliver the correct amounts through the tubing and into the patient's veins via the indwelling iv catheter.

There are a number of possible venipuncture sites on the human body, but for a peripheral iv catheter one of the most common sites where a catheter is inserted is the inside bend of the elbow called the antecubital fossa. An iv catheter in this region is notorious for causing problems with the flow or patency of iv catheters. A catheter in this area may allow flow while the arm is straight, but when the arm is flexed or bent it causes the internal catheter to kink (crimp, fold on itself, bend, or crease) which causes an “occlusion” or stop of flow of fluid through the lumen of the catheter. This results in a delay of the patient's iv treatment. During an occlusion the infusion pump is designed to alarm, the intended purpose is to alert the nurse to come fix the occlusion. Once the alarm has sounded, it will often have disturbed the sleep of the patient or other neighboring patients, as well as annoyed staff, visitors, or others in the area of the alarming infusion pump.

One common strategy to solve this problem of a kinked and occluded catheter is to encourage the patient to maintain an unflexed arm, but this is difficult for the general population of patients to do as it is quite difficult to hold an arm continually in a straight position, and some patients may not be alert enough to maintain an unflexed arm. Arm boards are sometimes used to restrain a patient's joint from flexing and thus maintaining a non-kinked catheter, however this does not solve the problem of discomfort that maintaining the extremity in a straight position can cause. It is also undesirable for the nurse to restrict/restrain any active freedom of movement of a patient.

As seen in FIGS. 1-4, by way of example only, a common intravenous catheter system and human arm (A) is shown, wherein an intravenous catheter (C), having a hub (H), is inserted at a cutaneous catheter insertion site (S) into a vein (V). In this case, and solely by way of example and not limitation, in this series of illustrations the vein (V) is an antecubital vein. The relative sizes and locations of the arm (A), catheter (C), hub (H), site (S) and vein (V) are intended for illustrative purposes only and are not necessarily shown to scale.

FIG. 2 shows an optimal relationship between the catheter (C), hub (H), arm (A) and vein (V), where the hub (H) and catheter (C) lie closely against the arm (A), and the catheter (C) lies in a near parallel relationship with the underlying vein (V). FIGS. 3 and 4 show two common scenarios that can induce failure of an intravenous system. As seen in FIG. 3, the hub (H) has become dislodged, no longer lying against the skin of the arm (A), and the catheter (C) is no longer in a near-parallel relationship with the underlying vein (V). As a result, a kink is seen forming near the insertion site (S). If the kink becomes severe enough, fluid will be unable to flow through the catheter (C). In FIG. 4, the hub (H) remains laying closely against the skin of the arm (A), but a bending of the arm (A)(in the exemplary case, a bending of the elbow), is causing the catheter (C) to begin forming a kink within the vein (V). Again, if the kink becomes sufficiently severe, fluid flow through the intravenous system will be compromised. Also, in both the scenarios depicted in FIGS. 3 and 4, the relative movement of the arm (A) and catheter (C) can lead to increased incidence of infection at the cutaneous catheter insertion site (S).

Various systems have been attempted to counter these problems, although they may generally be grouped into two types. One type attempts to counter the scenario seen in FIG. 3 by strongly securing the hub (H) and often adjacent portions of an intravenous apparatus to the patient, often with a very strong and complex structure. U.S. Pat. No. 3,194,235 is typical of this approach. Another type attempts to counter the scenario seen in FIG. 4 by partially or completely immobilizing the patient's limb. U.S. Pat. No. 3,196,870 is typical of this approach. Often, the approaches are combined. The approaches are often ineffectual, and also often severely increase patient discomfort and decrease mobility. The specification below teaches a new approach that solves the classical problems associated with cutaneous catheters in a novel series of ways. Overall the need for a device that prevents an indwelling iv catheter from kinking without decreasing the active range of motion of a patient's extremity is a long felt need in the hospital setting.

### SUMMARY OF THE INVENTION

The present invention includes a device and method to prevent occlusion of an indwelling peripheral intravenous catheter and to do so in a way that does not require immobilization of, or significant reduction in the range of motion of, the joint of the particular extremity. The cutaneous catheter anchoring device and method accomplish this by immobilizing multiple areas of the skin's surface relative to the indwelling iv catheter. The cutaneous catheter anchoring device is in close proximity to the indwelling iv catheter and maintains a smooth, rather than bent or creased, skin surface above the catheter thus preventing creasing or kinking of the catheter itself during times when the patient bends the extremity. There is little, to no, loss of active range of motion, and is much less restrictive, thereby much more comfortable for a patient than incorporating the use of traditional restraint devices and methods.

Embodiments of a cutaneous catheter anchoring device include a body, having a first width, a second width, a thickness, an edge, a top surface, a bottom surface, and a thickness. The body need not be round, and in some embodiments may have a second width that is greater than the first width to enhance the ability of the body to move transversely upon joint flexion. Embodiments of the body may be generally triangle, square, rectangular, pentagon, hexagon, heptagon, octagon, nonagon, decagon, pentadecagon, and icosagon in nature.

Numerous variations, modifications, alternatives, and alterations of the various preferred embodiments, processes, and methods may be used alone or in combination with one another as will become more readily apparent to those with skill in the art with reference to the following detailed description of the preferred embodiments and the accompanying figures and drawings.

### BRIEF DESCRIPTION OF THE DRAWINGS

Without limiting the scope of the present invention as claimed below and referring now to the drawings and figures:

FIG. 1 is a perspective view of the antecubital fossa area of a human arm (A), showing an intravenous catheter (C) inserted into an antecubital vein at an insertion site (S) and an intravenous catheter hub (H) overlying the antecubital fossa;

FIG. 2 is a schematic longitudinal cross section view taken along lines 2-2 of FIG. 1;

FIG. 3 is another schematic longitudinal cross section view taken along lines 2-2 of FIG. 1;

FIG. 4 is another schematic longitudinal cross section view taken along lines 2-2 of FIG. 1;

FIG. 5 is a top plan view of a part of an embodiment of the present invention;

FIG. 6 is a cross section view taken along lines 6-6 of FIG. 5;

FIG. 7 is a top plan view of a part of an embodiment of the present invention;

FIG. 8 is a cross section view taken along lines 8-8 of FIG. 7;

FIG. 9 is a top plan view of a part of an embodiment of the present invention;

FIG. 10 is a cross section view taken along lines 10-10 of FIG. 9;

FIG. 11 is a top plan view of a part of an embodiment of the present invention;

FIG. 12 is a cross section view taken along lines 12-12 of FIG. 11;

FIG. 13 is a medial perspective view of a human arm (A), showing an intravenous catheter (C) having a hub (H) inserted into an antecubital vein at a cutaneous catheter insertion site (S);

FIG. 14 is a medial perspective view of an embodiment of the present invention, with an intravenous apparatus removed for clarity;

FIG. 15 is a lateral perspective view of an embodiment of the present invention, with an intravenous apparatus again removed for clarity, and with a greater acute angulation of the elbow than as seen in FIG. 14;

FIG. 16 is a lateral perspective view of an embodiment of the present invention, with an intravenous apparatus again removed for clarity, and with a greater acute angulation of the elbow than as seen in FIG. 15;

FIG. 17 is a medial perspective view of an embodiment of the present invention, with an intravenous apparatus again shown removed for clarity, and with a degree of acute angulation of the elbow such as seen in FIG. 15;

FIG. 18 is a medial perspective view of an embodiment of the present invention, with an intravenous apparatus again shown removed for clarity, and with a degree of acute angulation of the elbow greater than that as seen in FIG. 17;

FIG. 19 is a longitudinal cross section view, showing an operating position of an embodiment of the present invention, overlaid on the arm (A) and catheter (C) as seen in FIG. 2;

FIG. 20 is a longitudinal cross section view, showing an operating position of another embodiment of the present invention, overlaid on the arm (A) and catheter (C) as seen in FIG. 2; and

FIG. 21 is a longitudinal cross section view, showing an operating position of another embodiment of the present invention, overlaid on the arm and catheter as seen in FIG. 2.

These illustrations are provided to assist in the understanding of the exemplary embodiments of a cutaneous catheter anchoring system as described in more detail below and should not be construed as unduly limiting the specification. In particular, the relative spacing, positioning, sizing and dimensions of the various elements illustrated in the drawings may not be drawn to scale and may have been exaggerated, reduced or otherwise modified for the purpose of improved clarity. Those of ordinary skill in the art will also appreciate that a range of alternative configurations have been omitted simply to improve the clarity and reduce the number of drawings.

### DETAILED DESCRIPTION OF THE INVENTION

The present invention includes a device and method to prevent occlusion of an indwelling peripheral intravenous catheter and to do so in a way that does not require immobilization of, or significant reduction in the range of motion of, the joint of the particular extremity. The cutaneous catheter anchoring device (10) and method accomplish this by immobilizing multiple areas of the skin's surface relative to the indwelling iv catheter. The device (10) serves to reinforce the skin and the catheter insertion site. The cutaneous catheter anchoring device (10) is in close proximity to the indwelling iv catheter and maintains a smooth, rather than bent or creased, skin surface above the catheter thus preventing creasing or kinking of the catheter itself during times when the patient bends the extremity. There is little, to no, loss of active range of motion, and is much less restrictive, thereby much more comfortable for a patient than incorporating the use of traditional restraint devices and methods.

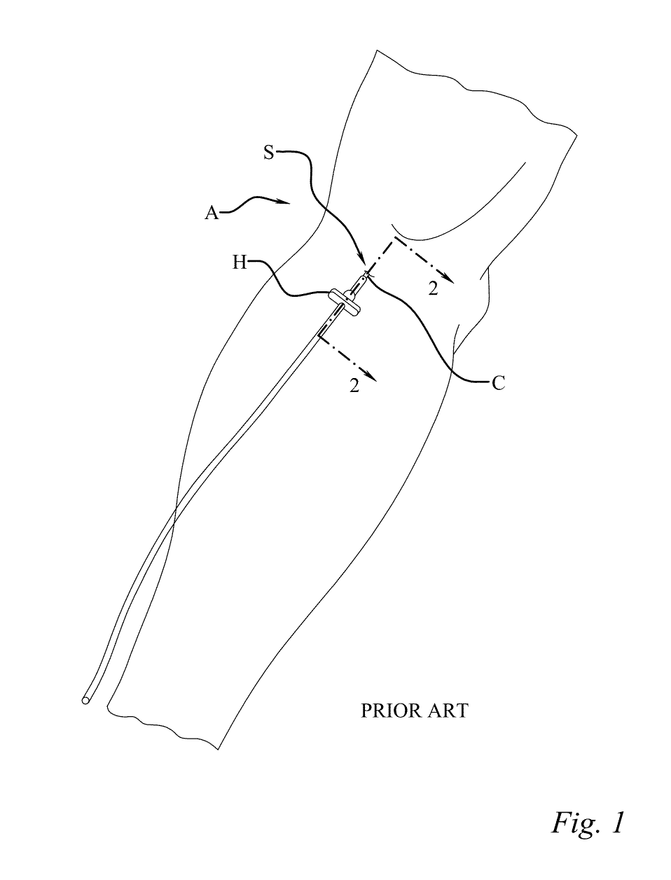

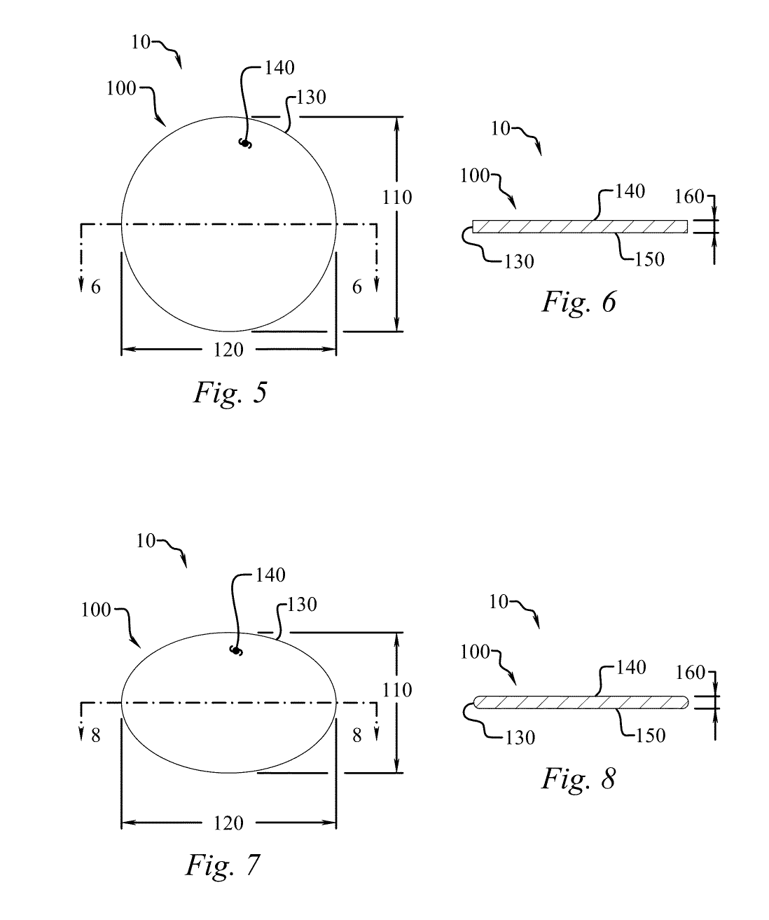

As seen in FIGS. 1-21, embodiments of a cutaneous catheter anchoring device (10) are disclosed. As seen well in FIGS. 5 and 6, the device (10) may include a body (100), having a first width (110), a second width (120), a thickness (160), an edge (130), a top surface (140), a bottom surface (160), and a thickness (160). The body (100) need not be round, and in some embodiments, as seen in FIGS. 7 and 8, may have a second width (120) that is greater than the first width (110), resulting in this case in a more or less oval or rectangular in shape, while in other embodiments the first width (110) and the second width (120) are substantially equal leading to substantially round or square shapes.

In one embodiment patient comfort is ensured, and interference with the catheter and/or hub is reduced, by avoiding sharp corners. For the purposes of this embodiment the term sharp corner means, with the body (100) laying on a flat surface and analyzing it in a top plan view orientation such as that seen in FIGS. 5 and 7, one in which two straight edges along the perimeter of the body (100) intersect to form an angle of less than 145 degrees between the edges. Alternatively, some embodiments may include straight sections of the perimeter, yet the straight sections do not physically intersect by incorporating a curved perimeter section, or fillet, having a radius of curvature of at least 0.125 inches, or a chamfer section having a chamfer edge length of at least 0.125 inches. Thus, further embodiments may include shapes that are generally polygon in nature provided any straight sections of the perimeter that would intersect to form an angle of less than 145 degrees are prevented from physically intersecting by incorporating the 0.125 inch fillet or chamfer edge length. Therefore, embodiments of the body (100) may be generally triangle, square, rectangular, pentagon, hexagon, heptagon, octagon, nonagon, decagon, pentadecagon, and icosagon in nature.

The body (100) is flat in one embodiment, but may be preformed to mimic the curvature of an insertion site (S). For instance in one such embodiment directed to the arm the body (100) may have a preformed shape with an angle of approximately 145-170 degrees, rather than a flat planar shape, to closely match the curvature of the arm. Further, in another embodiment, the bottom surface (160) may incorporate a recess, or recesses, that cooperates with the general shape of a catheter hub.

Patient comfort is improved, and potential interference with catheter and/or hub is reduced, in an embodiment wherein any straight sections of the perimeter are joined by a curved perimeter section, or fillet, having a radius of curvature of at least 0.25 inches, or a chamfer section having a chamfer edge length of at least 0.25 inches. Another embodiment improves patient comfort, while reducing potential interference, by only having perimeter edges with a radius of curvature that is at least twice the body thickness (160). An even further embodiment provides such benefits by avoiding ninety degree edges where the body edge (130) meets the body top surface (140) and/or the body bottom surface (150), as seen in FIG. 8. Such curved body edge(s) (130) ensure that compression of the body (100) against the skin during flexion of the extremity will not result in a tear or puncture to the dressing or skin. Taking this embodiment further, in another embodiment any straight sections of the body edge (130) joins a straight section of the body top surface (140) and/or body bottom surface (150) via a curved edge section, or fillet, having a radius of curvature of at least 25% of the body thickness (160), or a chamfer section having a chamfer edge length of at least 25% of the body thickness (160). Further, the embodiment of FIG. 8 incorporates a fillet at the body top surface (140) to body edge (130), as well as the body bottom surface (150) to body edge (130), wherein each fillet has a radius of curvature equal to substantially 50% of the body thickness (160). A further embodiment achieves the comfort and interference goals while still providing adequate rigidity of the body (100) by incorporating a fillet or chamfer at the body edge (130) to bottom surface (150) interface, while only having a chamfer or hard 90 degree edge at the body edge (130) to top surface (140) interface. As will be discussed in more detail later, adequate stiffness and rigidity of the body (100) ensures that it remains substantially in its original shape when subjected to the forces associated with a patient bending their joint near the insertion site (S); although some embodiments do allow for a predetermined amount of elastic deformation for enhanced patient comfort.

As previously mentioned, while the embodiments of FIGS. 5 and 7 illustrate entirely curved perimeters, such is not required. However, in one embodiment the perimeter has a perimeter length, and at least 50% of the perimeter length is curved; while in an even further embodiment at least 75% of the perimeter length is curved. Another embodiment also ensures patient comfort by ensuring that any straight sections of the perimeter are each individually less than 1.5″ long; while in a further embodiment these straight sections do not intersect at sharp corners, as previously defined; and in an even further embodiment all straight sections of the perimeter are less than 1.0″ long. Still further, one embodiment achieves the goals by having a total lineal length of perimeter straight sections that is less than 8″, while a further embodiment limits the total lineal length of perimeter straight sections to less than 5″.

Yet another embodiment does have an entirely curved perimeter wherein each point along the perimeter has a local radius of curvature, and the minimum local radius of curvature is at least 0.25″ and the maximum local radius of curvature is no more than 6″; while in an even further embodiment the minimum local radius of curvature is at least 0.5″ and the maximum local radius of curvature is no more than 3″. Again, such radius of curvature ensure patient comfort as they bend their joint under the device (10) while also reducing the likelihood of interference with the catheter (C) or hub (H). An even further embodiment improves patient comfort by incorporating a padded layer along the perimeter of the body edge (130). The total surface area of the body top surface (140) is preferably 0.79-7.7 square inches to satisfactorily to provide patient comfort over a wide range of common insertion sites (S) and joint sizes, while accommodating an improved range of motion, all the while ensuring reduced potential interference with catheter and/or hub. A further embodiment achieves these goals with the total surface area of the body top surface (140) of 1.77-7.07 square inches, while a further embodiment has the total surface area of the body top surface (140) of 2.41-5.94 square inches, and yet another embodiment has the total surface area of the body top surface (140) of 3.14-4.91 square inches. In one embodiment the body (100) is customizable by trimming it with a pair of scissors, or other cutting implement. Such customization allows the body (100) to be shaped to accommodate the age of the patient, status of the patient's subcutaneous connective tissue, and size of catheter (C) employed.

The modulus of elasticity of human skin ranges from about 15 to about 150 N/mm², with a mean of about 70 N/mm², or 10,150 lbf/in², at age 11 and declining to a mean of about 60 N/mm², or 8,702 lbf/in², at 95 years. The device (10) serves to reinforce the skin and the catheter insertion site. In one embodiment the stated goals are achieved when the maximum modulus of elasticity of the body (10) is at least ten times that of human skin, meaning the maximum modulus of elasticity of the body (10) is at least 87,000 lbf/in²; while a further embodiment the maximum modulus of elasticity of the body (10) is less than seventy-five times that of human skin, meaning the maximum modulus of elasticity of the body (10) is less than 652,500 lbf/in²; and an even further embodiment narrows the desired maximum modulus of elasticity of the body (10) to 100,000-500,000 lbf/in². In yet a further embodiment the maximum modulus of elasticity of the body (100) is at least ten times greater than the maximum modulus of elasticity of the underlayer (300). In one embodiment the maximum modulus of elasticity of the underlayer (300) is less than 10,000 lbf/in²; while in an even further embodiment the maximum modulus of elasticity of the underlayer (300) is less than 5,000 lbf/in².

Even further embodiments achieve the stated goals when formed of a material having a tensile strength at break of 3-24 MPa, while yet another embodiment falls within the range of 3-7 MPa. Further, the body (100) may be constructed of material having a percentage elongation at break of 200-400%. Additionally, the body (100) may be constructed of material having an impact strength of 60-200 J/m. Further, the maximum body thickness (160) is preferably 0.045″-0.250″, and one embodiment has a maximum body thickness (160) of 0.080″-0.125″. The disclosed material properties and geometric configurations, and relationships, provide the body with the flexural stiffness (EI) to provide the body (100) with a degree of give when placed over an insertion site (S) on a joint so that patient comfort is improved compared to a perfectly rigid body, while still possessing adequate stiffness to protect the catheter (C) and/or hub (H) and reduce the risk of kinking. In one embodiment the body (100) is formed of a thermoplastic material, which in a further embodiment is polyvinyl chloride (PVC) resin along with additives, such as plasticizers, stabilizers, pigments, and fillers. One particular embodiment includes a copolymer of vinyl chloride and vinyl acetate. One particular embodiment includes a rigid body (100) having a maximum modulus of elasticity of the body (10) of at least 1,000,000 lbf/in².

In some embodiments, the body (100) may be transparent to human vision, so that when applied over an inserted catheter, the catheter insertion site (S) may be more easily visualized. In still further embodiments, the bottom surface (150) may include a releasable adhesive applied to at least a portion of the bottom surface (150), preferably in a peel-and-stick configuration, so that the body (100) may be made self-adhering to the skin or sterile cover over the insertion site. In other embodiments, the body (100) itself may be the first sterile layer over the insertion site. In such an embodiment incorporating releasable adhesive, the adhesive may be applied to less than 75% of the surface area of the bottom surface (150) so that it does not adhere to the catheter (C) or hub (H), or even further the adhesive may be applied to 25-75 percent of the surface area of the bottom surface (150). While in a further embodiment the body (100) may include a topical antibiotic applied to at least a portion of the bottom surface (150), such that the body (100) may be integral to a dressing over the insertion site.

The device (10) may be a separate standalone product that is applied to the top of traditional site dressing, or such dressing may be incorporated into the device (10) to provide a single application product over the insertion site, which may be a multi-layer product or incorporate the body (100) and dressing in a single layer. In one embodiment the method of use is to affix the device (10) to the surface of the transparent film dressing of an already properly dressed iv site. Thus, the device (10) is to be affixed to an area of the transparent film dressing which is directly covering the skin, under which the catheterized area of the site's vein is located. The length of this protected area, which the device is directly above (with the transparent film dressing separating), is be greater than the approximate length of the catheter being used. In one embodiment the device (10) extends from the tip of the catheter back to the rigid, inflexible hub, which is the point where the flexible catheter terminates. The device (10) is located above the skin's surface on a functional iv site, and also covers at least a portion of the hub. Thus, the joint between the catheter and the hub will remain kink-free. The device (10) may also cover the area of the transparent film dressing that covers the stat-lock device, which is already adhesively anchored to the skin. In one embodiment, after proper attachment of the device (10), it can be seen that there is nearly a complete plane of adhesive engagement of the transparent film dressing to all areas immediately surrounding the actual catheter insertion point, which may include the adhesively engaged skin-to-stat-lock area, with the only exception being the hub which has no adhesive but is sufficiently surrounded by adhesively engaged skin-to-transparent film dressing areas. This nearly complete plane of adhesive fixation coupled to the bottom surface (150) of the body (100) to the top surface of the transparent film dressing, is what allows the patient to freely bend/flex the joint of the particular extremity without the worry of the catheter kinking and disrupting pharmaceutical iv therapy or causing the iv infusion pump to alarm as they frequently do when “occluded,” which often wakes patients and interrupts hospital staff. Another embodiment includes a further step including the application of a second transparent film dressing over the top of the body (100), which may also overlap the initial transparent film dressing. This added layer of adhesive engagement may be useful for active patients, meaning those with a tendency to bend their arms more frequently, as the added layer will help maintain the overall adhesive holding power longer as the adhesives may eventually loosen after repetitive bending. Thus, in another embodiment the top surface (140) may also include an adhesive layer, as this would again add still more strength toward the maintenance of a predetermined orientation of the skin surface under the forces of the arm bending against it.

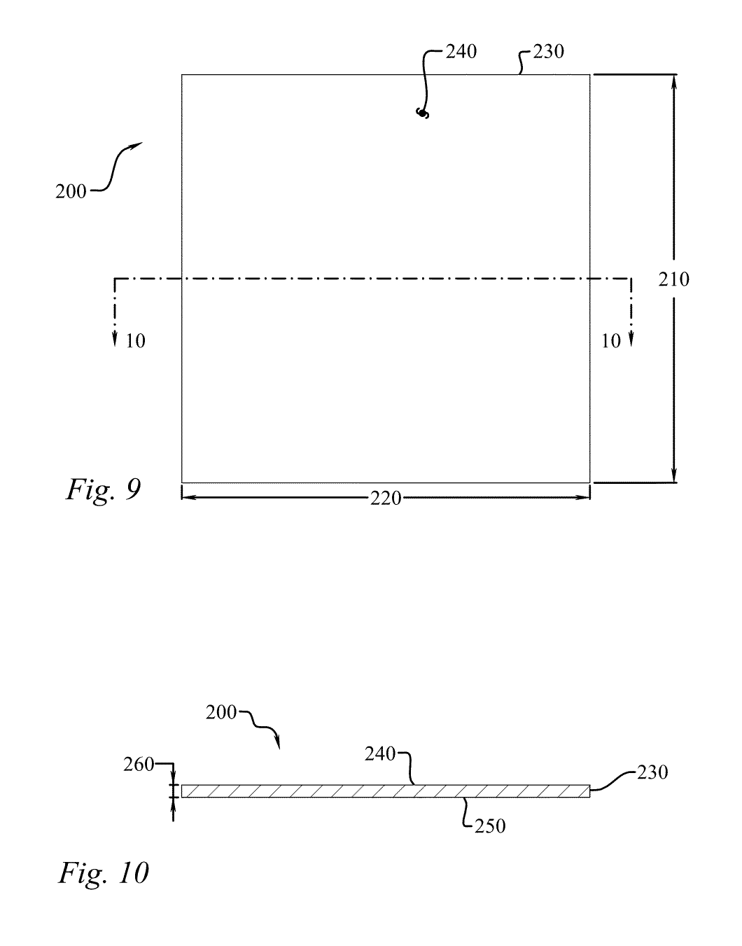

In further embodiments, the device (10) may be provided with a flexible overlayer (200) applied over the body (100) as seen well in FIGS. 9 and 10, and both anchoring the body (100) and helping seal a cutaneous catheter insertion site (S). The flexible overlayer (200) may have a first length (210), a second length (220), at least a circumferential edge (230), a top surface (240), a bottom surface (250) and a thickness (260). No particular shape is otherwise required, although a common shape might be rectangular, as illustrated by way of example only. However, one skilled in the art will see the utility of oval or other shaped overlayers (200) depending on the location and circumstances. The overlayer (200) may have a releasable adhesive applied to at least a portion of the bottom surface (250), so as to be incorporated as at least a part of a dressing for the site (S), and may overlie at least a portion of the body (100), helping to hold the body (100) in a proper position when the device (10) is in an operating position. The overlayer (200) may be formed of many of the various film-type dressings that are commonly in use.

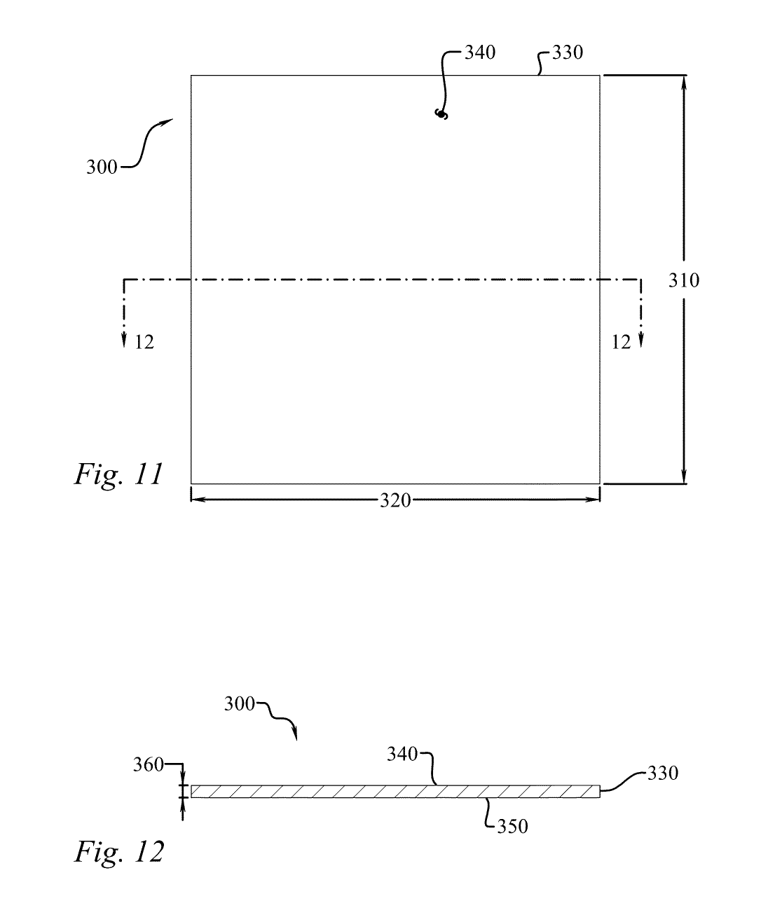

In other embodiments, as seen well in FIGS. 11, 12, and 14 the device (10) may include a flexible underlayer (300) applied under the body (100), i.e., between the body (100) and the skin. The flexible underlayer (300) may have a first length (310), a second length (320), an edge (330), a top surface (340), a bottom surface (350) and a thickness (360). The underlayer (300) may have a releasable adhesive applied to at least a portion of the bottom surface (350) so that the underlayer (300) may be self-adhering as part of a dressing over an insertion site (S). The underlayer (300) may underlie at least a portion of the body (100) and covers a cutaneous catheter insertion site (S) in an operating position. The thickness (360) of the underlayer (300) is preferably less than 50% of the body thickness (160), and the underlayer (300) may be formed of many of the various film-type dressings that are commonly in use. In some embodiments, the underlayer (300) may be transparent to human vision, so that an underlying cutaneous catheter insertion site (S) may be better visualized. In yet other embodiments, the underlayer bottom surface (350) may have a topical antibiotic applied to at least a portion of the bottom surface (350), such that the underlayer (300) may be incorporated as at least a part of a dressing over an insertion site (S). In one series of embodiments, the underlayer top surface (340) may have a releasable adhesive applied to at least a portion of the underlayer top surface (340), thereby allowing the underlayer (300) to be self-adhering to a body (100) placed on the top surface (340), as seen in FIG. 14.

These previous two embodiments may be seen together, such as wherein a flexible underlayer (300) has a releasable adhesive applied to at least a portion of the bottom surface (350) such that it may be self-adherent to the skin. Underlying a body (100) and covering a cutaneous catheter insertion site (S) in an operating position, the underlayer top surface (340) may have a releasable adhesive applied to at least a portion of the top surface (340), making it self-adhering to a body (100) placed on the top surface (340).

In some embodiments, the underlayer first length (310) may be equal to or greater than the body first width (110), while in others, underlayer second length (320) may be equal to or greater than the body second width (120), but no particular size relationship is required between the underlayer (200), the overlayer (300) and the body (100), and each may cover a different amount of surface area.

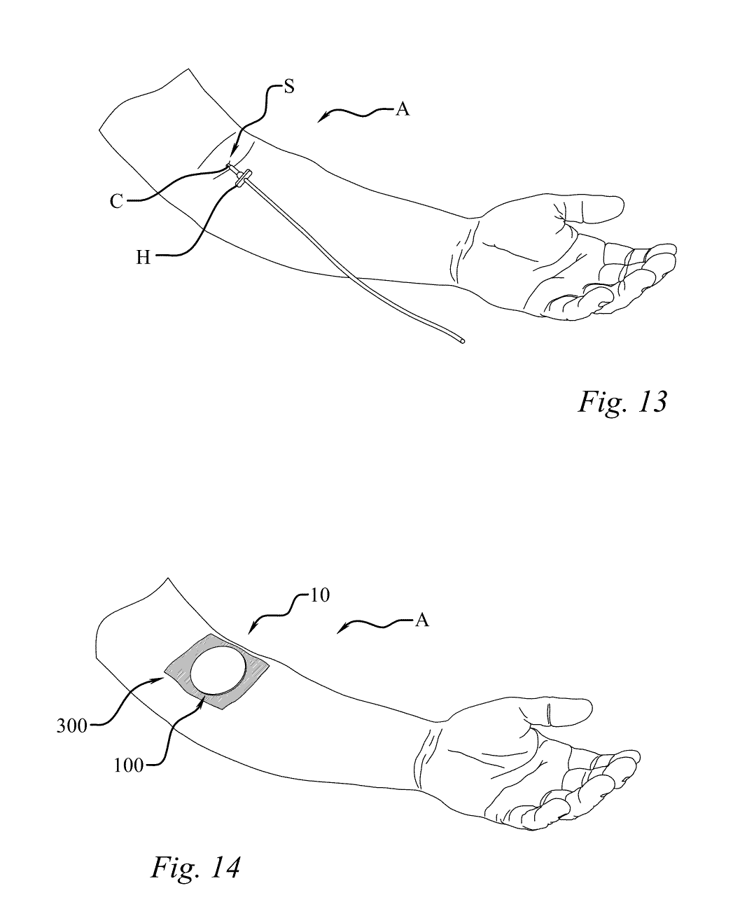

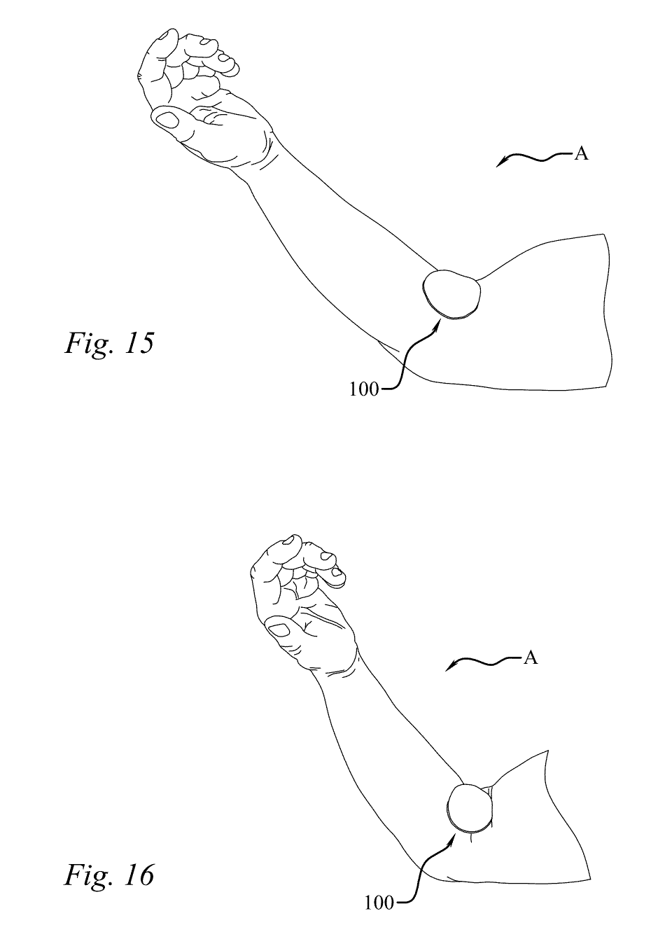

The body (100) of the present invention effects a number of salutary effects on an inserted intravenous catheter (C) and hub (H), and this may be seen well in the series shown in FIGS. 13-18 of the behavior of a body relative to different arm positions. When the arm is straightened, i.e., the elbow is not bent, the body (100) lies relatively flat over an insertion site (s), as seen in FIGS. 13-14. In this, and all other positions, the body (100) makes a first contribution by serving as a mechanical shield over the insertion site (S). But importantly, other effects begin as the arm begins to become angulated, as seen in FIGS. 15-18. Firstly, the body (100) inhibits a fully acute angulation of the elbow, while relatively little affecting less acute angulations, thereby minimizing the potential for catheter (C) kinking, while maximizing patient comfort. As previously noted, the body (100) has sufficient stiffness that it will not fold thereby inhibiting fully acute angulation, however it may possess some flexibility, or give, to further patient comfort.

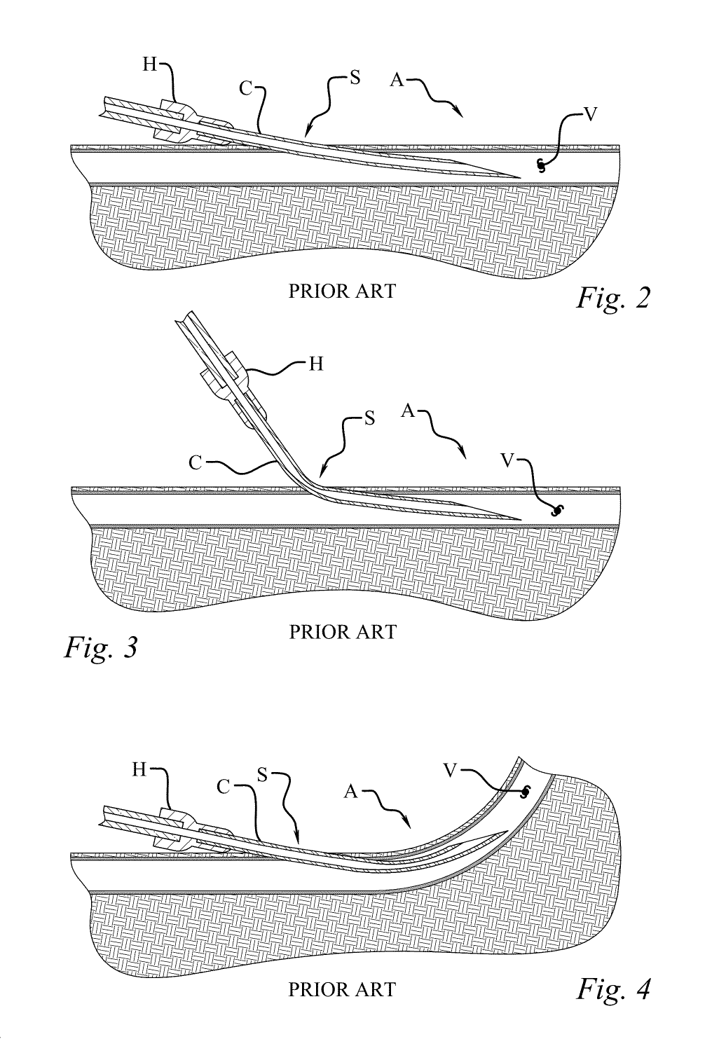

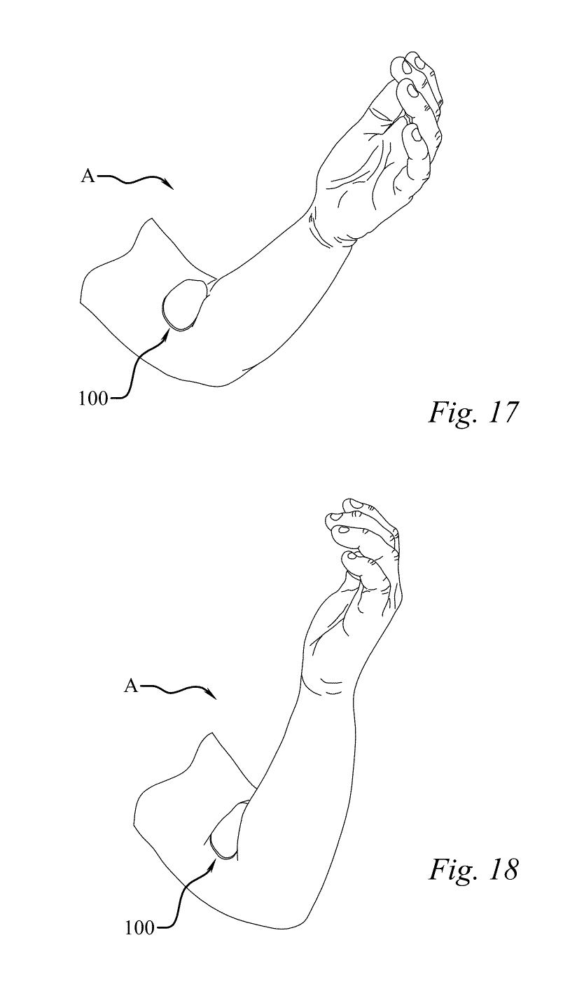

At the same time, angulation of the arm tends to displace the body (100) either slightly laterally, seen in FIGS. 15 and 16, or medially, as seen in FIGS. 17 and 18. This displacement does not cause a displacement of the body (100) relative to the skin surface, to which it is affixed, but is permitted by a medial or lateral displacement of the skin and the connective tissue underlying the skin. Because the body holds the catheter (C) and hub (H) in the ideal spatial relationship to an underlying vein, as seen in FIGS. 2 and 19-21, the risk of kinking and disruption is reduced.

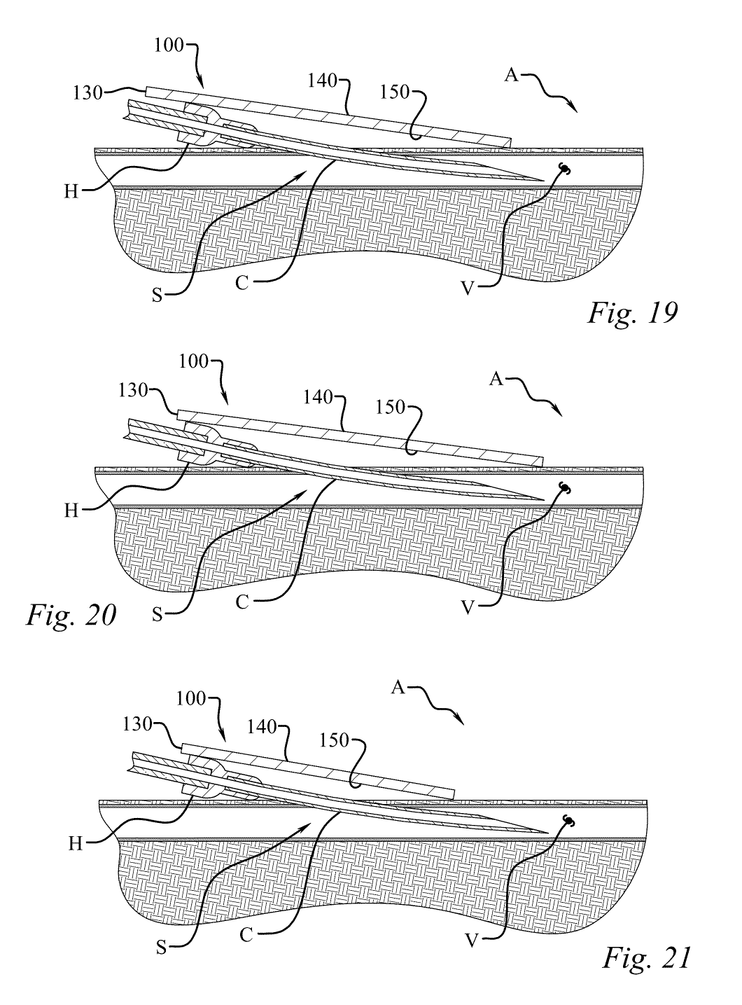

In one series of embodiments, seen well in FIG. 19, the body (100) in an operating position may completely cover a hub (H) of an underlying inserted intravenous catheter (C) when an intersection point of an imaginary line drawn along the first width (110) and an imaginary line drawn along the second width (120), on the top surface (140) is placed directly over a cutaneous catheter insertion site (S), or, in simple terms, when the body is centered over the insertion site (S). Because the body (100) has some degree of resilience, and because of the not-to-scale nature of FIGS. 19-21, the body (100) actually lies flatter against the skin than may be apparent from the figures.

In another series of embodiments, seen well in FIG. 20, the body (100) in an operating position may completely cover a full length of an underlying inserted intravenous catheter (C) and a hub (H) measured from a most proximal point of the hub (H) towards a most distal point of the catheter (C). (Throughout this specification, “proximal” shall mean most distant from a catheter discharge orifice, while “distal” shall mean closest to a catheter discharge orifice.) This may be perhaps the most secure configuration for the body (100), but will also result in the largest body (100), which may be a factor in patient comfort.

In yet another series of embodiments, seen well in FIG. 21, the body (100) in an operating position may cover about three quarters of a length of an underlying inserted intravenous catheter (C) and a hub (H) measured from a most proximal point of the hub (H) towards a most distal point of the catheter (C). This configuration may represent a compromise between patient comfort and catheter (C) security.

Therefore, combining various features as discussed above, in some embodiments, a catheter anchoring device (10) may include a body (100) having a first width (110), a second width (120), a thickness (160), at least a circumferential edge (130), a top surface (140), a bottom surface (150) and a thickness (160). The body (100) in an operating position may completely cover a hub (H) of an underlying inserted intravenous catheter (C) having a cutaneous catheter insertion site (S) when an intersection point of an imaginary line drawn along the first width (110) and an imaginary line drawn along the second width (120), on the top surface (140) is placed directly over the cutaneous catheter insertion site (S), or in other words, when the body (100) is centered over the insertion site (S), as seen well in FIG. 19.

In yet another series of embodiments, seen well in FIG. 21, a cutaneous catheter anchoring device (10) may include a body (100) having a first width (110), a second width (120), a thickness (160), at least a circumferential edge (130), a top surface (140), a bottom surface (150), and a thickness (160). The body (100) in an operating position may cover about three quarters of a length of an underlying inserted intravenous catheter (C) and a hub (H) measured from a most proximal point of a hub (H) to a most distal point of the catheter (C). A flexible underlayer (300) may underlie the body (100), the underlayer (300) having a first length (310), a second length (320), at least a circumferential edge (330), a top surface (340), a thickness (360) and a bottom surface (350) having a releasable adhesive applied to at least a portion of the bottom surface (350), and the underlayer (300) may cover a cutaneous catheter insertion site (S).

Now, a further method to prevent occlusion of an indwelling peripheral intravenous catheter and to do so in a way that does not require immobilization of, or significant reduction in the range of motion of, the joint of the particular extremity will be disclosed. Here the method for immobilizing an area of skin relative to an indwelling catheter in the vicinity of an insertion site at the antecubital fossa, while allowing flexion of an elbow joint in the vicinity of a catheter insertion site to reduce the risk of occlusion during joint flexion, comprising the steps of: a) releasably coupling a lateral skin area of at least 0.25 in², located laterally of the insertion site, and a medial skin area of at least 0.25 in², located medially of the insertion site, to a cutaneous catheter anchoring device (10); b) wherein the cutaneous catheter anchoring device (10) covers a portion of a catheter hub and extends longitudinally beyond a catheter discharge orifice, and wherein the cutaneous catheter anchoring device (10) shifts transversely upon flexion of the elbow joint and maintains the relationship to the coupled lateral skin area and medial skin area thereby shifting the insertion site transversely with the cutaneous catheter anchoring device (10) to prevent kinking of the catheter; and c) wherein the cutaneous catheter anchoring device (10) includes a body (100) having a first width (110), a second width (120), a circumferential edge (130) defining a perimeter, a top surface (140), a bottom surface (150), and a thickness (160), wherein the top surface (140) has a surface area of 0.79-7.70 in², the thickness (160) is 0.045-0.250″, and the perimeter has a total lineal length of straight perimeter sections that is less than 8″. The method may further include the step of releasably coupling a distal skin area of at least 0.25 in², located longitudinally beyond the catheter discharge orifice, to the cutaneous catheter anchoring device (10). One skilled in the art will appreciate that the size of the skin area coupled to the cutaneous catheter anchoring device (10) ensures that the skin, and therefore the catheter, moves transversely with the device (10) during flexion, while spreading out the load on the skin and avoid deleterious effects of large point loads, particularly in elderly patients. In fact, in a further embodiment the areas are increased such that the lateral skin area is at least 0.50 in² having a longitudinal lateral skin length of at least 0.50″, and the medial skin area is at least 0.50 in² having a longitudinal medial skin length of at least 0.50″. An even further embodiment further increases the areas such that the lateral skin area is at least 0.75 in² having a longitudinal lateral skin length of at least 0.50″, and the medial skin area is at least 0.75 in² having a longitudinal medial skin length of at least 0.50″. Still further, another embodiment achieves the goals when the distal skin area of at least 0.50 in², combined with a lateral skin area of at least 0.75 in² having a longitudinal lateral skin length of at least 0.50″, and a medial skin area of at least 0.75 in² having a longitudinal medial skin length of at least 0.50″.

Further, the method may include the step of also releasably coupling a distal skin area of at least 0.25 in², located longitudinally beyond the catheter discharge orifice, to the cutaneous catheter anchoring device (10), wherein the distal skin area has a transverse distal skin length of at least 0.125″. Coupling the skin to the cutaneous catheter anchoring device (10) on at least 3 sides of the catheter further ensures safe movement of the skin and catheter during joint flexion. The step of releasably coupling the lateral skin area and the medial skin area to the cutaneous catheter anchoring device (10) may further include the step of covering the insertion site with a sterile flexible underlayer (300) attached to the skin, and attaching the body (100) to an exterior of the flexible underlayer (300). Yet a further embodiment may include the step of trimming the body (100) to create a custom patient specific shape to accommodate their size and anticipated range of motion. In fact, in one embodiment the body (100) includes at least one scoreline such that the body may be snapped along the scoreline to customize the size of the body (100). In a variation of this embodiment the at least one scoreline allows the medical personal to reduce the total surface area of the body top surface (140) by at least 25%, whereas a further embodiment allows the medical personal to reduce the total surface area of the body top surface (140) by at least 50%.

Numerous alterations, modifications, and variations of the embodiments disclosed herein will be apparent to those skilled in the art and they are all anticipated and contemplated to be within the spirit and scope of the instant invention. For example, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the present invention are described herein, it is to be understood that the practice of such additional modifications and variations and the equivalents thereof, are within the spirit and scope of the invention as defined in the following claims.

1. A cutaneous catheter anchoring device (10), comprising,
a body (100) having a first width (110), a second width (120), a circumferential edge (130) defining a perimeter, a top surface (140), a bottom surface (150), and a thickness (160);
wherein the top surface (140) has a surface area of 0.79-7.70 in²;
wherein the thickness (160) is 0.045-0.250″; and
wherein the perimeter has a total lineal length of straight perimeter sections that is less than 8″.

2. The device according to claim 1, wherein straight perimeter sections do not intersect and are joined by a curved perimeter section having a radius of curvature of at least twice the thickness (160).

3. The device according to claim 1, wherein at least 50% of the perimeter length is curved.

4. The device according to claim 3, wherein the entire perimeter length is curved.

5. The device according to claim 4, wherein each point along the perimeter has a local radius of curvature and the minimum local radius of curvature is 0.25″ and the maximum local radius of curvature is 6.0″.

6. The device according to claim 1, wherein the top surface (140) has a surface area of 2.41-5.94 in².

7. The device according to claim 6, wherein the thickness (160) is 0.080-0.125″ and the maximum modulus of elasticity of the body (100) is at least 87,000 lbf/in².

8. The device according to claim 2, wherein the perimeter includes at least two straight perimeter section and all straight perimeter sections have individual lineal lengths of less than 1.5″.

9. The device according to claim 2, wherein the edge (130) and the bottom surface (150) are joined by a curved edge section having a radius of curvature that is at least 25% of the thickness (160).

10. The device according to claim 8, wherein straight perimeter sections do not intersect and are joined by a curved perimeter section having a radius of curvature of at least 0.25″.

11. A method for immobilizing an area of skin relative to an indwelling catheter in the vicinity of an insertion site at the antecubital fossa, while allowing flexion of an elbow joint in the vicinity of a catheter insertion site to reduce the risk of occlusion during joint flexion, comprising the steps of:
a) releasably coupling a lateral skin area of at least 0.25 in², located laterally of the insertion site, and a medial skin area of at least 0.25 in², located medially of the insertion site, to a cutaneous catheter anchoring device (10) to reinforce the skin and the catheter insertion site;
b) wherein the cutaneous catheter anchoring device (10) covers a portion of a catheter hub and extends longitudinally beyond a catheter discharge orifice, and wherein the cutaneous catheter anchoring device (10) shifts transversely upon flexion of the elbow joint and maintains the relationship to the coupled lateral skin area and medial skin area thereby shifting the insertion site transversely with the cutaneous catheter anchoring device (10) to prevent kinking of the catheter; and
c) wherein the cutaneous catheter anchoring device (10) includes a body (100) having a first width (110), a second width (120), a circumferential edge (130) defining a perimeter, a top surface (140), a bottom surface (150), and a thickness (160), wherein the top surface (140) has a surface area of 0.79-7.70 in², the thickness (160) is 0.045-0.250″, and the perimeter has a total lineal length of straight perimeter sections that is less than 8″.

12. The method of claim 11, further including the step of releasably coupling a distal skin area of at least 0.25 in², located longitudinally beyond the catheter discharge orifice, to the cutaneous catheter anchoring device (10), wherein the distal skin area has a transverse distal skin length of at least 0.125″.

13. The method of claim 11, wherein the lateral skin area is at least 0.50 in² having a longitudinal lateral skin length of at least 0.50″, and the medial skin area is at least 0.50 in² having a longitudinal medial skin length of at least 0.50″.

14. The method of claim 11, wherein the step of releasably coupling the lateral skin area and the medial skin area to the cutaneous catheter anchoring device (10) further includes the step of covering the insertion site with a sterile flexible underlayer (300) attached to the skin, and attaching the body (100) to an exterior of the flexible underlayer (300).

15. The method of claim 11, wherein the maximum modulus of elasticity of the body (100) is at least 87,000 lbf/in².

16. The method of claim 15, wherein the maximum modulus of elasticity of the body (100) is less than 652,000 lbf/in², and the thickness (160) is 0.080-0.125″.

17. The method of claim 14, wherein the maximum modulus of elasticity of the body (100) is at least ten times greater than the maximum modulus of elasticity of the underlayer (300).

18. The method of claim 11, wherein the shape of the cutaneous catheter anchoring device (10) promotes the transverse shift upon flexion of the elbow joint by having a curved perimeter over at least 50% of the perimeter length.

19. The method of claim 11, wherein the entire perimeter length is curved and each point along the perimeter has a local radius of curvature and the minimum local radius of curvature is 0.25″ and the maximum local radius of curvature is 3.0″.

20. The method of claim 19, wherein the second width (120) is greater than the first width (110).
